(19) United States
(12) Patent Application Publication
(10) Pub. No.: US 2010/0159546 A1
(43) Pub. Date: Jun. 24, 2010

(54) METABOLIC ENGINEERING OF YEASTS FOR THE PRODUCTION OF 1-BUTANOL

(76) Inventors: Aristos Aristidou, Highland, CO (US); Arlene M. Fosmer, Minnetonka, MN (US); Catherine Asleson Dundon, Englewood, CO (US); Brian J. Rush, Minneapolis, MN (US)

Correspondence Address: Gary C. Cohn, PLLC, 215 E. 96TH ST., #19L, New York, NY, 10128, US

(21) Appl. No.: 12/593,842

(22) PCT Filed: Mar. 27, 2008
(86) PCT No.: PCT/US08/58406

§ 371 Date: Jan. 27, 2010

Related U.S. Application Data
(60) Provisional application No. 60/909,410, filed on Mar. 30, 2007.

(51) Int. Cl. C12P 7/16; C12N 1/19

(52) U.S. Cl. 435/160; 435/254.2; 435/254.21; 435/254.22; 435/254.23

(57) ABSTRACT

The present invention provides genetically modified yeast cells, and methods of using those yeast cells, to produce 1-butanol. The yeast cell can be selected from the genera Saccharomyces, Candida, Pichia, Kluyveromyces, Issatchenkia, Yarrowia, Rhodotorula, Hansenula, Schizochytrium, or Thraustochytrium. The yeast cell of the invention overexpresses at least one enzyme that catalyzes one or more butanoate pathways selected from the group consisting of pyruvate to acetyl-CoA, acetyl-CoA to acetoacetyl-CoA, acetoacetyl-CoA to 3-hydroxybutanoyl-CoA, 3-hydroxybutanoyl-CoA to crotonoyl-CoA, crotonoyl-CoA to butyryl-CoA to butyraldehyde, and butyraldehyde to 1-butanol. Some embodiments overexpress enzymes that are endogenous to the bacterium Clostridium acetobutylicum. The genetically modified yeast cell can further be subjected to other desired genetic changes, such as deletion or disruption of one or more native PDC genes.

This application claims priority from U.S. Provisional Application No. 60/909,410, filed 30 Mar. 2007. This invention relates to engineered microorganisms and fermentation processes for the production of 1-butanol from one or more carbon sources.

1-butanol is a four-carbon alcohol (C₄H₉OH) that can be utilized as a liquid transportation fuel (alone or in combination with other liquid fuels). 1-butanol's energy content is comparable to that of standard gasoline. 1-butanol can be shipped through existing fuel pipelines due to its good miscibility with gasoline and relatively low miscibility with water. 1-butanol has a very low vapor pressure and a high flash point. In general, the properties of 1-butanol make it an attractive oxygenated liquid fuel.

1-butanol is also an important industrial chemical. 1-butanol is currently used as a feedstock chemical in the plastics industry and as a food-grade extractant in the food and flavor industry. 1-butanol also has a widespread use as an industrial solvent.

Originally produced by fermentation starting about a century ago, 1-butanol manufacture shifted to petrochemical routes in the 1950s as the price of petroleum-derived feedstocks dropped. Virtually all of the 1-butanol in use today is produced petrochemically. Petrochemical processes to produce 1-butanol include propylene hydroformylation with syngas; crotonaldehyde hydrogenation; and Reppe synthesis of propylene, carbon monoxide, and water. As petroleum costs rise, fermentation routes to 1-butanol can become more attractive, particularly as developments are made that increase the biocatalyst and fermentation performance.

The fermentation of carbohydrates to acetone, 1-butanol, and ethanol by bacterial solventogenic Clostridia is well known. More specifically, it is known in the art that Clostridium acetobutylicum can be used in acetone-butanol-ethanol (ABE) fermentation. For example, U.S. Pat. No. 5,192,673 describes an improved fermentation process for producing high levels of 1-butanol using a mutant strain designated C. acetobutylicum ATCC 55025. U.S. Pat. No. 6,358,717 describes a method of producing 1-butanol using a fermentation process that employs a mutant strain of C. beijerinckii. U.S. Pat. No. 5,063,156 describes a process including multistage continuous fermentation followed by batch fermentation with carefully chosen temperatures for each fermentation step, combined with an asporogenic strain of C. acetobutylicum. In that specification, it is disclosed that a high (60-120 g/L) carbohydrate substrate concentration yields over 20 g/L 1-butanol.

The production of 1-butanol in bacteria is limited by severe product inhibition. 1-Butanol at a concentration of 1% can significantly inhibit cell growth and the fermentation process. Consequently, the 1-butanol concentration in bacterial ABE fermentations is usually lower than about 15 g/L, in order to reduce the inhibition effect. This significant problem associated with ABE fermentation was first described by Chaim Weizmann in 1912 (Jones and Woods, Microbiol. Rev. 50:484-524, 1986). Therefore, in order to make 1-butanol production economical in a fermentation process, a need exists for new methods of increasing the titers of 1-butanol produced during fermentation.

Another problem with fermentation processes to produce 1-butanol is that bacteria are unable to synthesize some of the amino acids or proteins they need to grow and metabolize sugars efficiently. As a result, bacteria often must be fed a somewhat complex package of nutrients. This need increases the direct expense required to operate the fermentation. The increased complexity of the broth makes it more difficult to recover the fermentation product in reasonably pure form, so increased operating and capital costs are incurred to recover the product.

Given these significant challenges for 1-butanol fermentation, it would be desirable to develop a more efficient process for producing 1-butanol from sugar substrates. Preferably, the fermentation process can achieve high volumetric and specific productivities. The process preferably can produce a high yield of 1-butanol from the fermentation substrate and can produce high 1-butanol titers. The process preferably can be operated under microaerobic and especially anaerobic conditions. The process preferably can be operated using a simplified defined media.

The present invention provides yeast cells to produce 1-butanol. In one aspect, this invention is a genetically modified yeast cell having an active metabolic pathway from a fermentable sugar to 1-butanol. In some embodiments, the active metabolic pathway includes a pathway from pyruvate to 1-butanol. Such a pathway can include the reactions a) pyruvate to acetyl-CoA, b) acetyl-CoA to acetoacetyl-CoA, c) acetoacetyl-CoA to 3-hydroxybutanoyl-CoA, d) 3-hydroxybutanoyl-CoA to crotonoyl-CoA, d) crotonoyl-CoA to butyryl-CoA, f) butyryl-CoA to butyraldehyde, and g) butyraldehyde to 1-butanol. For purposes of this invention, “to have an active metabolic pathway” means that the cell produces active enzymes necessary to catalyze each reaction in the pathway and therefore produces, under fermentation conditions and in the presence of a fermentable sugar, the product of the pathway in measureable yields.

In some embodiments, the genetically modified yeast cell described above further has deletion or disruption of a native metabolic pathway from pyruvate to ethanol. This can be achieved through the deletion or disruption of one or more native pyruvate decarboxylase (PDC) genes, and/or the deletion or disruption of one or more native alcohol dehydrogenase ADH genes.

The present invention is also a method of producing 1-butanol by culturing the yeast cell of the invention in the presence of a fermentable carbon source.

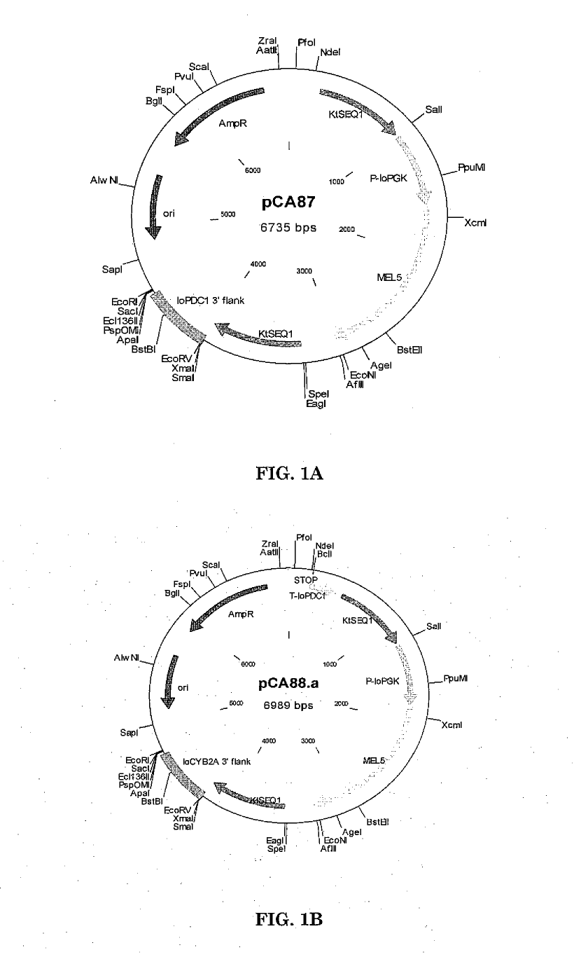

FIG. 1A is a diagram depicting the pCA87 plasmid.

FIG. 1B is a diagram depicting the pCA88.a plasmid.

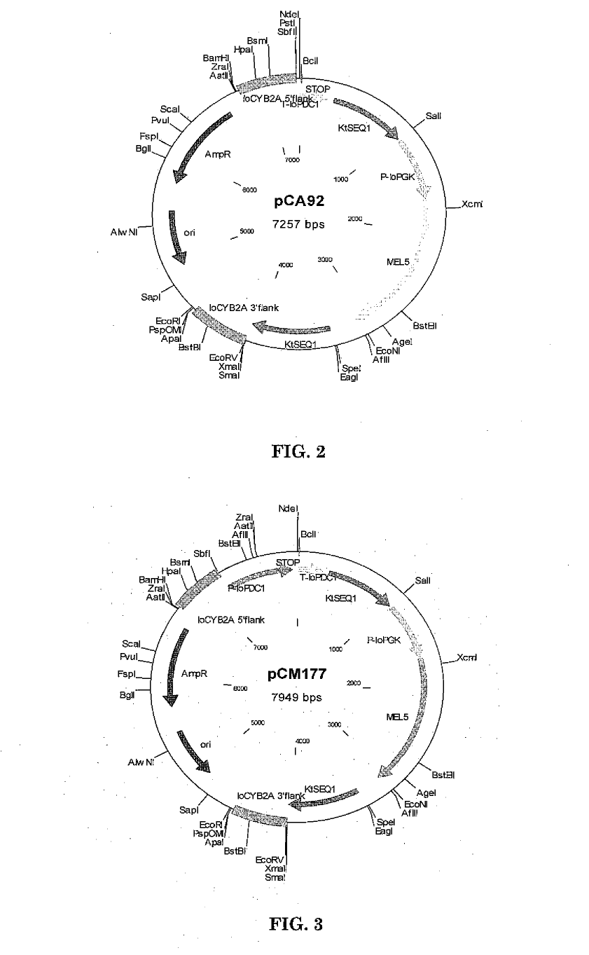

FIG. 2 is a diagram depicting the pCA92 plasmid.

FIG. 3 is a diagram depicting the pCM177 plasmid.

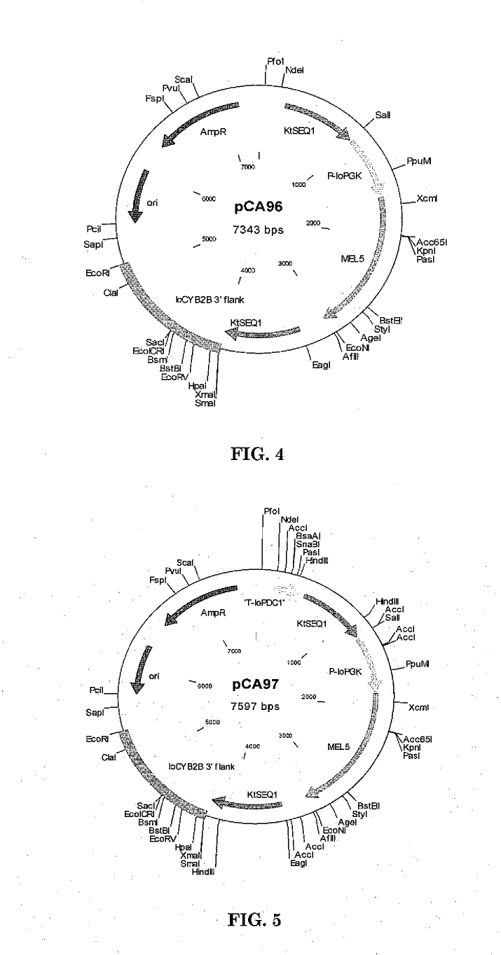

FIG. 4 is a diagram depicting the pCA96 plasmid.

FIG. 5 is a diagram depicting the pCA97 plasmid.

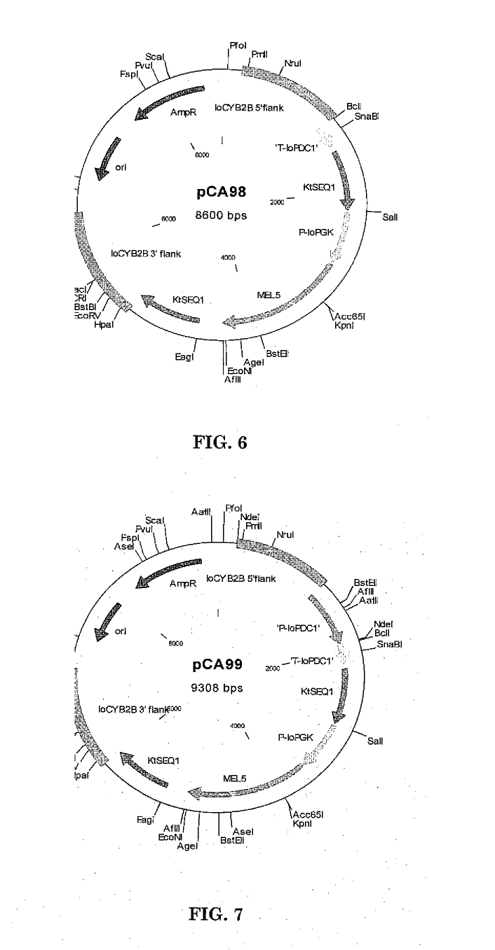

FIG. 6 is a diagram depicting the pCA98 plasmid.

FIG. 7 is a diagram depicting the pCA99 plasmid.

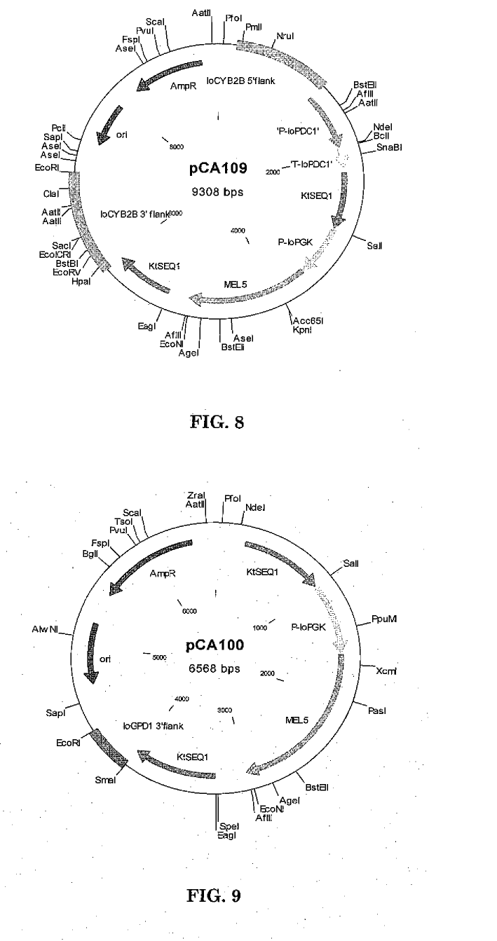

FIG. 8 is a diagram depicting the pCA109 plasmid.

FIG. 9 is a diagram depicting the pCA100 plasmid.

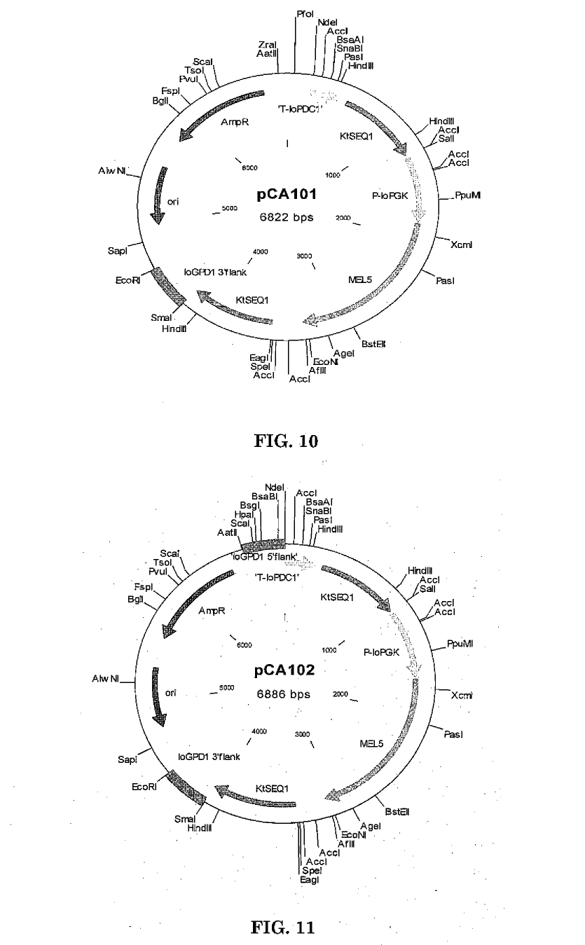

FIG. 10 is a diagram depicting the pCA101 plasmid.

FIG. 11 is a diagram depicting the pCA102 plasmid.

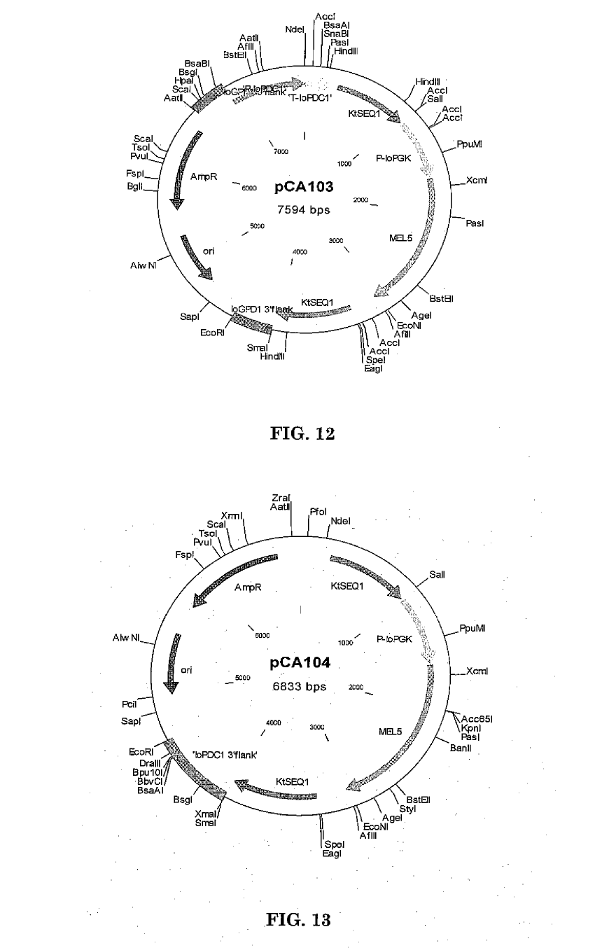

FIG. 12 is a diagram depicting the pCA103 plasmid.

FIG. 13 is a diagram depicting the pCA104 plasmid.

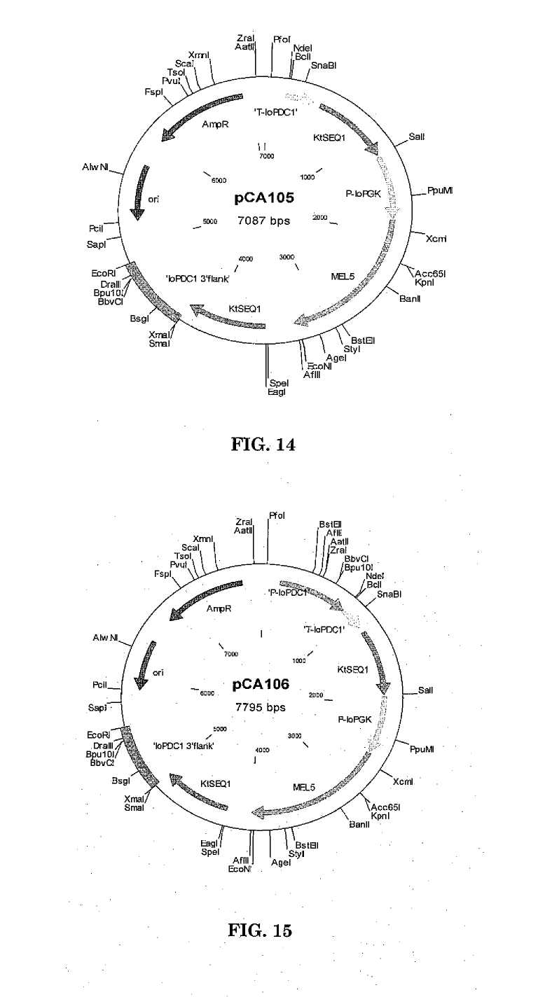

FIG. 14 is a diagram depicting the pCA105 plasmid.

FIG. 15 is a diagram depicting the pCA106 plasmid.

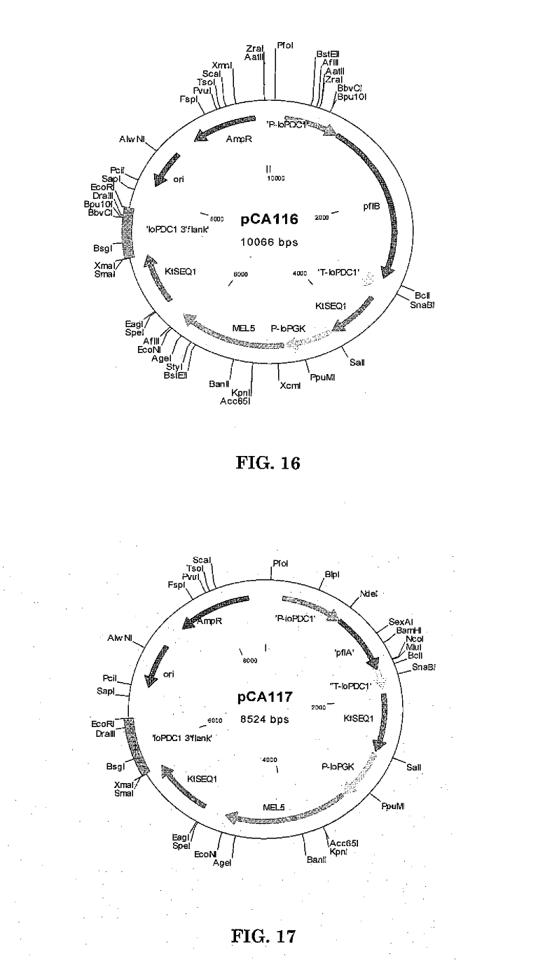

FIG. 16 is a diagram depicting the pCA116 plasmid.

FIG. 17 is a diagram depicting the pCA117 plasmid.

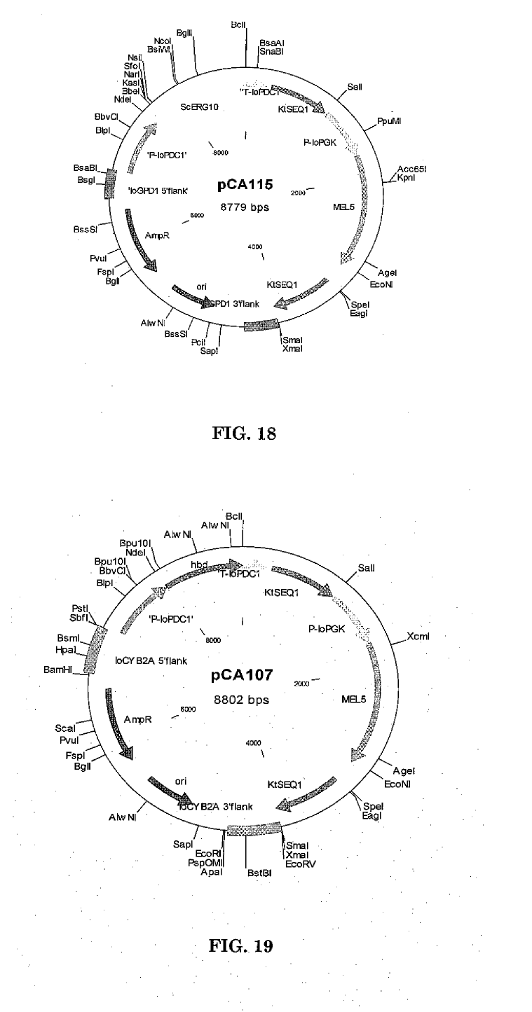

FIG. 18 is a diagram depicting the pCA115 plasmid.

FIG. 19 is a diagram depicting the pCA107 plasmid.

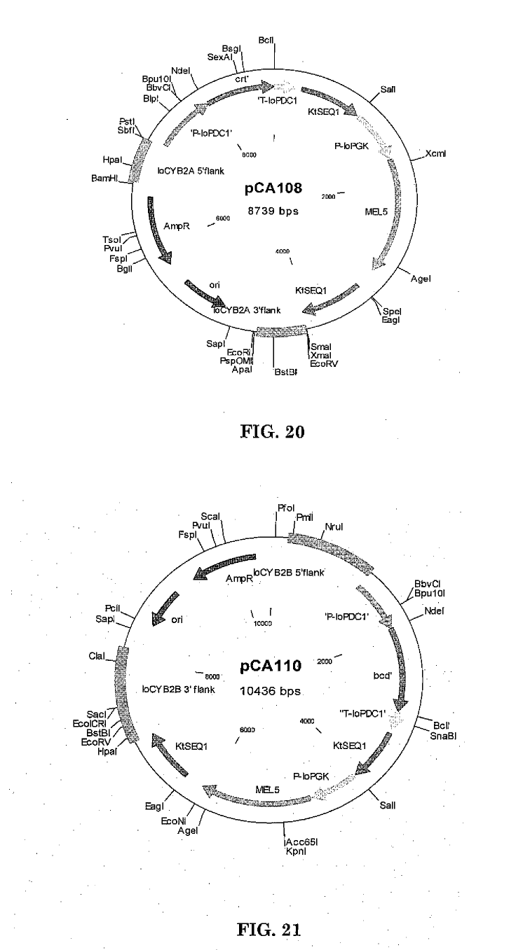

FIG. 20 is a diagram depicting the pCA108 plasmid.

FIG. 21 is a diagram depicting the pCA110 plasmid.

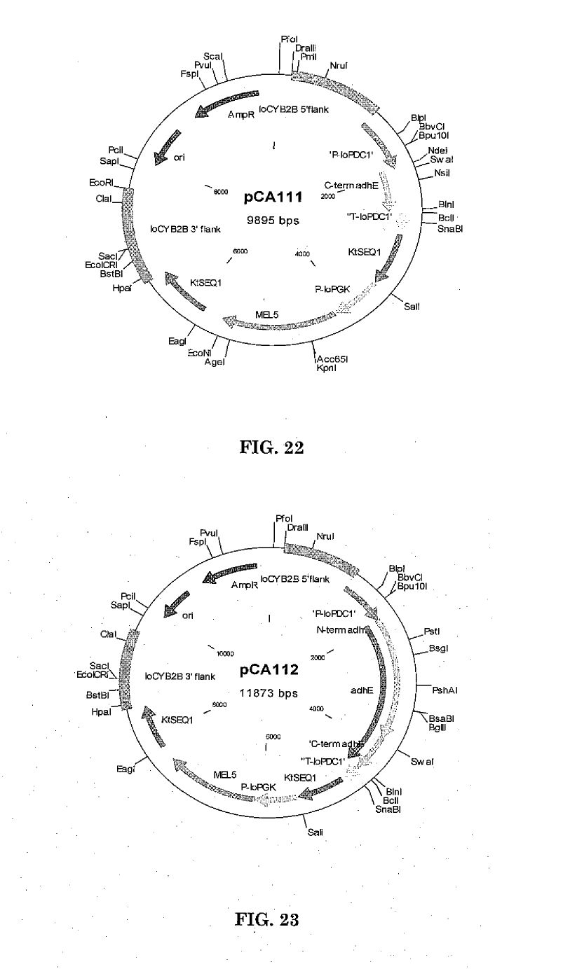

FIG. 22 is a diagram depicting the pCA111 plasmid.

FIG. 23 is a diagram depicting the pCA112 plasmid.

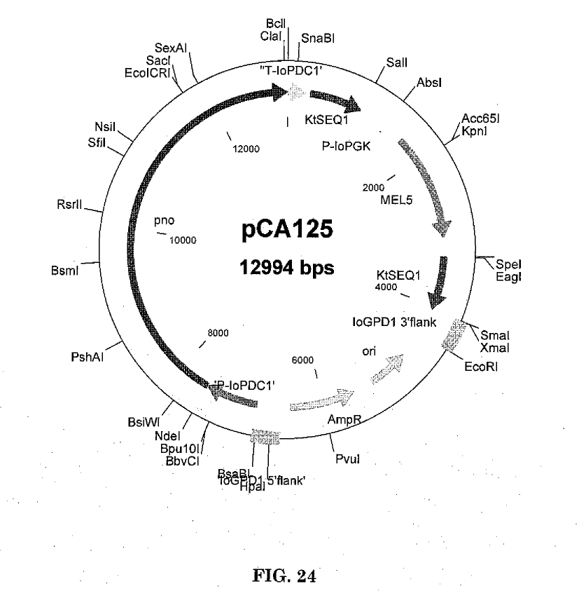

FIG. 24 is a diagram depicting the pCA125 plasmid.

According to the present invention, yeasts are metabolically engineered to convert fermentable carbon sources to 1-butanol. Most yeasts are natural ethanol producers but do not generally produce longer-chain alcohols such as 1-butanol. No known yeast contains a functional pathway to produce 1-butanol, so at least part of the necessary metabolic pathway will need to be added to any yeast chosen for the purposes of the present invention.

Any host yeast may be employed for the purposes of the present invention. Candidate yeasts can be selected on various relevant criteria before, during, or after attempting to engineer in a pathway to 1-butanol. These secondary criteria include relative tolerance to 1-butanol, glycolytic rates, specific growth rates, thermotolerance, tolerance to biomass hydrolysate inhibitors, overall process robustness, and so on. These criteria can be evaluated in host cells, engineered cells, cells that have been evolved, cells that have been subjected to mutagenesis and selection, or cells that have otherwise been modified and screened.

Suitable yeast cells preferably can synthesize their needed amino acids or proteins from inorganic nitrogen compounds. They often grow and ferment well in so-called “defined” media, which are simplified, often less expensive and present fewer difficulties in product (1-butanol) recovery operations. Suitable yeast cells also can preferably ferment under relatively harsh conditions, such as those that may be encountered when using biomass hydrolysates as sugar feedstocks.

In some embodiments, the yeast is selected from the genera Saccharomyces, Candida, Pichia, Kluyveromyces, Issatchenkia, Yarrowia, Rhodotorula, Hansenula, Schizochytrium, or Thraustochytrium. Some exemplary yeast species include Saccharomyces cerevisiae, Hansenula ofunaensis, H. polymorphs, H. anomala, Schizochytrium limacinum, Issatchenkia orientalis, Thraustochytrium striatum, T. roseum, T. aureum, Candida sonorensis, Kluyveromyces marxianus, K. lactis, and K. thermotolerans. Suitable strains of K. marxianus and C. sonorensis include those described in WO 00/71738 A1, WO 02/42471 A2, WO 03/049525 A2, WO 03/102152 A2 and WO 03/102201A2. A suitable strain of I. orientalis is ATCC strain 32196.

The genetically modified yeast cell of the invention contains at least one active, exogenous gene that encodes for an enzyme that catalyzes at least one reaction in a metabolic pathway from a fermentable carbon source to 1-butanol. In the context of this invention, “exogenous” refers to genetic material (e.g., a gene, promoter or terminator) that is not native to the host strain. The term “native” is used herein with respect to genetic materials that are found (apart from individual-to-individual mutations which do not affect function) within the genome of wild-type cells of the host cell.

A preferred genetically modified yeast cell contains an active metabolic pathway from pyruvate to 1-butanol, and contains at least one active, exogenous gene that encodes for an enzyme that catalyzes at least one reaction in the metabolic pathway from pyruvate to 1-butanol.

One possible metabolic pathway proceeds through the C₄ compound oxaloacetate, which is derived from the carboxylation of pyruvate.

Preferably, the yeast cell has an active metabolic pathway from pyruvate to 1-butanol that includes the reactions: a) pyruvate to acetyl-CoA, b) acetyl-CoA to acetoacetyl-CoA, c) acetoacetyl-CoA to 3-hydroxybutanoyl-CoA, d) 3-hydroxybutanoyl-CoA to crotonoyl-CoA, d) crotonoyl-CoA to butyryl-CoA, f) butyryl-CoA to butyraldehyde, and g) butyraldehyde to 1-butanol. This metabolic pathway is sometimes referred to below as the “butanoate” pathway. In these preferred embodiments the yeast cell contains at least one active, exogeneous gene that encodes for an enzyme that catalyzes at least one of reactions a)-g). In addition, the yeast cell may overexpress one or more native genes that produce enzyme(s) that catalyze one or more of reactions a)-g).

By “overexpress”, it is meant that the gene produces significantly more of an active enzyme that the cell produces in a wild-type strain. Overexpression can be accomplished, for example, by increasing the number of copies of the gene, by increasing the binding strength of the promoter region, or by other mechanisms. Forced evolution or mutagenesis can be used to produce yeast strains which overexpress one or more native genes.

A “butanoate” pathway for metabolizing pyruvate to 1-butanol is present in bacteria such as Clostridium acetobutylicum or C. beijerinckii and may be present in some fungus protists. The butanoate pathway present in these organisms is representative of a pathway from pyruvate to 1-butanol that can be introduced into the yeast cell of the invention. The butanoate pathway that is present in these organisms is summarized in the following table. In addition, suitable sources of genes of the various types, which can be used to transform a host cell in accordance with this invention are indicated in the table.

| | E.C. No. | | |
|---|---|---|---|
| | (C. aceto- | | |
| Reaction Step | butylicum) | Enzyme Name | Representative Source Genes |
| a) pyruvate to | 2.3.1.54 | pyruvate-formate lyase or | E. coli pflA (SEQ. ID. NO. 23) |
| acetyl-CoA | or | pyruvate dehydrogenase | E. coli pflB (SEQ ID. NO. 19) |
| | 1.2.1.51 | | Piromyces sp. E2 pflB (SEQ. ID. |
| | | | NO. 54 (partial); |
| | | | E. gracilis pno (SEQ. ID. NO. |
| | | | 49) |
| b) 2 acetyl-CoA to | 2.3.1.9 | acetyl-CoA | S. cerevisiae ERG10 |
| acetoacetyl-CoA | | acetyltransferase | (SEQ. ID. NO. 27) |
| c) acetoacetyl-CoA to | 1.1.1.157 | 3-hydroxybutyryl-CoA | C. acetobutylicum hbd |
| 3-hydroxybutanoyl- | | dehydrogenase | (SEQ. ID. NO. 31) |
| CoA | | | |
| d) 3-hydroxybutanoyl- | 4.2.1.55 | 3-hydroxybutyryl-CoA | C. acetobutylicum crt |
| CoA | | dehydratase | (SEQ. ID. NO. 35) |
| to crotonoyl-CoA | | | |
| e) crotonoyl-CoA to | 1.3.99.2 | butyryl-CoA | C. acetobutylicum bcd |
| butyryl-CoA | or | dehydrogenase | (SEQ. ID. NO. 39) |
| | 1.3.1.44 | or | E. gracilis ter (SEQ. ID. NO. 53) |
| | | trans-2-enyl-CoA | |
| | | reductase | |
| f) butyryl-CoA to | 1.2.1.10 | acetaldehyde | C. acetobutylicum adhE |
| butyraldehyde | | dehydrogenase | (SEQ. ID. NO. 43) |
| g) butyraldehyde to | 1.1.1.— | 1-butanol dehydrogenase | C. acetobutylicum adhE |
| 1-butanol | | | (SEQ. ID. NO. 43) |

As illustrated in the foregoing table, the butanoate pathway first converts the C₃ pyruvate to a C₂ compound (acetyl-CoA) and CO₂. Two acetyl-CoA molecules are then fused together to form the C₄ compound 3-acetoacetyl-CoA, which then undergoes a series of chemical modifications (mainly dehydrogenation and dehydration reactions) in order to form the C₄ compound I-butanol which can be exported outside the cell.

The overall theoretical stoichiometry for the butanoate pathway is C₆H₁₂O₆═C₄H₁₀O+2 CO₂+H₂O from glucose and C₅H₁₀O₅=5/6 C₄H₁₀O+10/6 CO₂+5/6 H₂O from xylose or arabinose. According to these overall reactions, the maximum theoretical yield of 1-butanol on glucose is 1 mol/mol or 0.41 g/g glucose, and the maximum theoretical yield of 1-butanol from xylose or arabinose is 5/6 mol/mol or 0.41 g/g.

The host cell is transformed to introduce into its genome one or more functional genes that produce enzymes which catalyze at least one step in the metabolic pathway to 1-butanol. The host cell is transformed with genes as necessary to provide the cell with a complete active metabolic pathway to 1-butanol. The host cell may naturally produce enzymes that catalyze one or more of the reactions in that metabolic pathway. For example, wild-type yeast cells such as Saccharomyces cerevisiae are believed to produce functional enzymes which can catalyze reactions a), b) and g) of the butanoate pathway. In cases such as these, in which the host cell naturally produces some of the needed enzymes, it may be necessary only to transform the host cell to introduce exogenous genes to produce those enzymes which are missing and therefore needed to complete the metabolic pathway. Accordingly, the genes that must be supplied in a particular case will depend on which genes are native to the host cell. In the case of S. cerevisiae, it may be sufficient only to introduce genes which produce enzymes that catalyze steps c)-f) of the butanoate pathway. Even if the wild-type cells produce an enzyme needed in the butanoate pathway, it may be necessary or desirable to supplement the activity of that gene by introducing additional copies of one or more native genes, by introducing exogenous genes that produce enzymes that catalyze the same reaction or which more specifically catalyze the particular reaction in the metabolic pathway to 1-butanol, or by otherwise overexpressing the native gene.

For fully genome-sequenced yeasts, whole-genome stoichiometric models can be used to better understand which enzymes are lacking, given a desired pathway for 1-butanol production. For example, whole-genome stoichiometric models for S. cerevisiae are described in, for example, Hjersted et al., “Genome-scale analysis of Saccharomyces cerevisiae metabolism and ethanol production in fed-batch culture,” Biotechnol. Bioeng. 2007; and Famili et al., “Saccharomyces cerevisiae phenotypes can be predicted by using constraint-based analysis of a genome-scale reconstructed metabolic network,” Proc. Natl. Acad. Sci. 2003, 100(23):13134-9.

Genes needed to complete the metabolic pathway include those which catalyze at least one reaction along the metabolic pathway. Native genes of these types can be obtained from bacterial, fungal, yeast or mammalian sources. In addition, other genes, which encode enzymes having the needed catalytic activity and which are at least 50%, 60%, 70% or 80% identical at the amino acid level to enzymes encoded by one or more of the aforementioned native genes, can be used. The genes obtained from any of these sources may be subjected to mutagenesis if necessary to provide a coding sequence starting with the usual eukaryotic starting codon (ATG), to enhance their catalytic activity, or for other purposes.

Identities scores of amino acid sequences of DNA, RNA or proteins are, for purposes of this invention, computed using the BLAST version 2.2.13 algorithm with default parameters. The BLAST software is available from the National Center for Biological Information, Bethesda, Md.

The exogenous genes needed to complete the metabolic pathway can be donated from organisms such as C. acetobutylicum that contain a complete butanoate pathway. In this case, the entire metabolic pathway can be obtained from a single donor species. Alternatively, the exogenous genes can be donated by an organism which contains only a part of the butanoate pathway. Certain anaerobic fungi and protists contain some of the needed genes.

Suitable genes for step a) of the butanoate pathway include bacterial, yeast, protist and fungal pyruvate formate lyase and pyruvate dehydrogenase genes. Examples of such genes include E. coli pflA (identified as SEQ. ID. NO. 23), E. coli pflB (identified as SEQ ID. NO. 19), Piromyces sp. E2 pflB (partial sequence identified as SEQ. ID. NO. 54) and E. gracilis pno (identified as SEQ. ID. NO. 49) genes, or other functional genes that are at least 50% identical, preferably at least 75% identical to any of such genes. The E. coli pflA, E. coli pflB, Piromyces sp. E2 pflB and E. gracilis pno genes encode for proteins having the protein sequences identified as SEQ. ID. NO. 24, SEQ. ID. NO. 20, SEQ. ID. NO. 55 and SEQ. ID. NO. 50, respectively. Genes that encode for functional enzymes having an amino acid sequence at least 50% identical, preferably at least 75% identical to any of SEQ ID NOs. 20, 24, 50 or 55 are suitable.

Suitable genes for step b) of the butanoate pathway include yeast 2 acetyl-CoA to acetoacetyl-CoA genes. An example of such a gene is S. cerevisiae ERG10 (identified as SEQ. ID. NO. 27), or other functional genes that are at least 50% identical, preferably at least 75% identical to that gene. The S. cerevisiae ERG10 gene encodes for a protein having the protein sequence identified as SEQ. ID. NO. 28. Genes that encode for functional enzymes having an amino acid sequence at least 50% identical, preferably at least 75% identical to SEQ ID NO. 28 are suitable.

Suitable genes for step c) of the butanoate pathway include bacterial 3-hydroxybutyryl-CoA dehydrogenase genes. An example of such a gene is the C. acetobutylicum hbd (identified as SEQ. ID. NO. 31) gene, or other functional genes that are at least 50% identical, preferably at least 75% identical to that gene. The C. acetobutylicum hbd gene encodes for a protein having the protein sequence identified as SEQ. ID. NO. 32. Genes that encode for functional enzymes having an amino acid sequence at least 50% identical, preferably at least 75% identical to SEQ ID NO. 32 are suitable.

Suitable genes for step d) of the butanoate pathway include bacterial 3-hydroxybutanoyl-CoA to crotonoyl-CoA genes. An example of such a gene is the C. acetobutylicum crt (identified as SEQ. ID. NO. 35) gene, or other functional genes that are at least 50% identical, preferably at least 75% identical to that gene. The C. acetobutylicum crt gene encodes for a protein having the protein sequence identified as SEQ. ID. NO. 36. Genes that encode for functional enzymes having an amino acid sequence at least 50% identical, preferably at least 75% identical to SEQ ID NO. 36 are suitable.

Suitable genes for step e) of the butanoate pathway include bacterial crotonoyl-CoA to butyryl-CoA genes. Examples of such a gene are the C. acetobutylicum bcd (identified as SEQ. ID. NO. 39) and E. gracilis ter (identified as SEQ. ID. NO. 52) genes, or other functional genes that are at least 50% identical, preferably at least 75% identical to either of those two genes. The C. acetobutylicum bcd gene encodes for a protein having the protein sequence identified as SEQ. ID. NO. 40. The E. gracilis ter genen encodes for a protein having the protein sequence identified as SEQ. ID. NO. 53. Genes that encode for functional enzymes having an amino acid sequence at least 50% identical, preferably at least 75% identical to SEQ. ID. NO. 40 or SEQ. ID. NO. 53 are suitable.

Suitable genes for steps f) and g) of the butanoate pathway include bacterial dehydrogenase genes such as the C. acetobutylicum adhE gene (identified as SEQ. ID. NO. 43), or other functional genes that are at least 50% identical, preferably at least 75% identical to that gene. The C. acetobutylicum adhE gene encodes for a protein which catalyzes both the butyryl-CoA to butyraldehyde and the butyraldehyde to 1-butanol reactions. The enzyme produced by that gene has the protein sequence identified as SEQ. ID. NO. 44. Genes that encode for functional enzymes having an amino acid sequence at least 50% identical, preferably at least 75% identical to SEQ ID NO. 44 are suitable.

Each exogenous gene that is introduced into the yeast cell of the invention is under the transcriptional control of one or more promoters and one or more terminators, both of which are functional in the modified yeast cell. As used herein, the term “promoter” refers to an untranslated sequence located upstream (i.e., 5′) to the translation start codon of a structural gene (generally within about 1 to 1000 bp, preferably 1-500 bp, especially 1-100 bp) and which controls the start of transcription of the structural gene. Similarly, the term “terminator” refers to an untranslated sequence located downstream (i.e., 3′) to the translation finish codon of a structural gene (generally within about 1 to 1000 bp, more typically 1-500 base pairs and especially 1-100 base pairs) and which controls the end of transcription of the structural gene.

Promoters and terminator sequences may be native to the host cell or exogenous to the cell. Useful promoter and terminator sequences include those that are highly identical (i.e., have an identities score of 90% or more, especially 95% or more, most preferably 99% or more) in their functional portions compared to the functional portions of one or more promoter and terminator sequences, respectively, that are native to the host cell—particularly when the insertion of the exogenous gene is targeted at a specific site in the cell's genome.

The exogenous genes may be integrated randomly into the host cell's genome or inserted at one or more targeted locations. The use of native (to the host cell) promoters and terminators, together with their respective upstream and downstream flanking regions, can permit the targeted integration of the exogenous gene or genes into specific loci of the host cell's genome, and for simultaneous integration of the exogenous gene and deletion of another native gene. Examples of targeted locations include the loci of a gene that is desirably deleted or disrupted, such as a native pyruvate decarboxylase (PD)C or a native alcohol dehydrogenase (ADH) gene. Integration of a butanoate gene at the PDC or ADH locus may be accomplished with or without deletion or disruption of the native PDC or ADH gene, but it is generally preferred to disrupt or delete the PDC or ADH gene, so the modified cell produces less ethanol.

The transformation of the host cells to introduce the needed genes is accomplished in one or more steps via the design and construction of appropriate vectors and transformation of the host cell with those vectors. Electroporation and/or chemical (such as calcium chloride- or lithium acetate-based) transformation methods can be used. Methods for transforming yeast strains to insert an exogenous gene and to delete native genes are described in WO 99/14335, WO 00/71738, WO 02/42471, WO 03/102201, WO 03/102152 and WO 03/049525. The vectors can either be cut with particular restriction enzymes or used as circular DNA.

In general, a vector is prepared that contains one or more genes to be inserted and associated promoter and terminator sequences. The vector may contain restriction sites of various types for linearization or fragmentation. Vectors may further contain a backbone portion (such as for propagation in E. coli) many of which are conveniently obtained from commercially available yeast or bacterial vectors.

It is usually desirable that the vector includes a functional selection marker cassette. When a single deletion construct is used, the marker cassette resides on the vector downstream (i.e., in the 3′ direction) of the 5′ sequence from the target locus and upstream (i.e., in the 5′ direction) of the 3′ sequence from the target locus. Successful transformants will contain the selection marker cassette, which imparts to the successfully transformed cell some characteristic that provides a basis for selection.

A “selection marker gene” is one that encodes a protein needed for the survival and/or growth of the transformed cell in a selective culture medium. Typical selection marker genes encode proteins that (a) confer resistance to antibiotics or other toxins (such genes as, for example, zeocin (Streptoalloteichus hindustanus ble bleomycin resistance gene), G418 (kanamycin-resistance gene of Tn903) or hygromycin (aminoglycoside antibiotic resistance gene from E. coli)), (b) complement auxotrophic deficiencies of the cell (such as, for example, amino acid leucine deficiency (K. marxianus LEU2 gene) or uracil deficiency (e.g., K. marxianus or S. cerevisiae URA3 gene)); (c) enable the cell to synthesize critical nutrients not available from simple media, or (d) confer ability for the cell to grow on a particular carbon source (such as a MEL5 gene from S. cerevisiae, which encodes the alpha-galactosidase (melibiase) enzyme and confers the ability to grow on melibiose as the sole carbon source). Preferred selection markers include the zeocin resistance gene, G418 resistance gene, a MEL5 gene and a hygromycin resistance gene.

The selection marker cassette will further include promoter and terminator sequences, operatively linked to the selection marker gene, and which are operable in the host cell. One suitable type of promoter is at least 50%, 70%, 90%, 95% or 99% identical to a promoter that is native to a yeast gene. A more suitable type of promoter is at least 50%, 70%, 90%, 95% or 99% identical to a promoter for a gene that is native to the host cell. Particularly useful promoters include promoters for pyruvate decarboxylase (PDC1), phosphoglycerate kinase (PGK), xylose reductase (XR), xylitol dehydrogenase (XDH), L-(+)-lactate-cytochrome c oxidoreductase (CYB2), translation elongation factor-1 (TEF1) and translation elongation factor-2 (TEF2) genes, especially from the respective genes of the host cell. An especially useful promoter includes the functional portion of a promoter for a PDC1, PGK, TEF1 or TEF2 gene native to the host cell, or a sequence that is at least 80%, 85%, 90% or 95% identical to such a PDC1, PGK, TEF1 or TEF2 promoter.

One suitable type of terminator is at least 50%, 70%, 90%, 95% or 99% identical to a terminator for a gene that is native to a yeast cell. The terminator may be at least 50%, 70%, 90%, 95% or 99% identical to a terminator for a gene that is native to the host cell. Particularly useful terminators include terminators for pyruvate decarboxylase (PDC1), xylose reductase, (XR), xylitol dehydrogenase (XDH), L-lactate:ferricytochrome c oxidoreductase (CYB2) or iso-2-cytochrome c (CYC) genes, or a terminator from the galactose family of genes in yeast, particularly the GAL10 terminator. An especially preferred terminator includes a functional portion of a terminator for a GAL10 gene native to the host cell, or a sequence that is at least 80%, 85%, 90% or 95% identical to such a terminator.

Successful transformants can be selected for in known manner, by taking advantage of the attributes contributed by the marker gene, or by other characteristics (such as ability to produce 1-butanol, inability to produce ethanol (if the pyruvate to ethanol metabolic path is disrupted), or ability to grow on specific substrates) contributed by the inserted genes. Screening can be performed by PCR or Southern analysis to confirm that the desired insertions and deletions have taken place, to confirm copy number and to identify the point of integration of genes into the host cell's genome. Activity of the enzyme encoded by the inserted gene and/or lack of activity of enzyme encoded by the deleted gene can be confirmed using known assay methods.

When the various genetic modifications have been completed, the transformed cell has an active metabolic pathway from the carbon source to 1-butanol, meaning that there is sufficient enzymatic activity for each step in the pathway such that the carbon source can be converted to 1-butanol thorough the individual pathways discussed above. The transformed yeast cell contains at least one exogenous gene, and will typically contain from two to seven exogenous genes to complete the metabolic pathway from the carbon source to 1-butanol. The transformed cell can contain multiple genes for any step or steps in the metabolic pathway. When the transformed cell contains multiple butanoate genes, the individual genes may be copies of the same gene, or may include copies of two or more different genes. Multiple copies of an exogenous gene may be integrated at a single locus (so they are adjacent to each other), or at several loci within the host cell's genome.

In some embodiments, the cell has additional genetic modifications that reduce or eliminate its ability to ferment the carbon source to produce ethanol. This is preferably done by deleting or disrupting a native metabolic pathway from pyruvate to ethanol. A native pathway from pyruvate to ethanol can be deleted or disrupted by deleting or disrupting one or more pyruvate decarboxylase (PDC) genes that are native to the wild-type yeast cells, and/or deleting or disrupting alcohol dehydrogenase (ADH) gene or genes that are native to the host yeast cell. By “deletion or disruption” of a metabolic pathway, it is meant that the pathway is either rendered completely inoperative, or else its activity is reduced by at least 75%, preferably at least 90% relative to the wild-type cell. Activity of a pathway may be reduced by reducing the amount of active enzyme that is produced, or by reducing the activity of the enzyme that is produced. By “deletion or disruption” of a gene, it is meant that the entire coding region of the gene is eliminated (deletion), or the gene, its promoter, and/or its terminator region is modified (such as by deletion, insertion, or mutation) so that the gene no longer produces an active enzyme, or produces an enzyme with severely reduced (at least 75% reduced, preferably at least 90% reduced) activity. Deletion or disruption can generally be accomplished by genetic-engineering methods, forced evolution, mutagenesis and selection, or other types of screening.

It is also possible to simultaneously delete a native PDC or ADH gene (or other gene) and simultaneously insert one or more genes, including one or more of the genes need to complete the metabolic pathway to 1-butanol, into the locus of the deleted gene. This is done by constructing a deletion construct that contains two non-continguous portions of the locus of the target gene (and including at least a portion of the target gene) and a cassette containing the gene to be inserted, together with promoter and terminator sequences, located between the non-continguous sequences. The construct may also contain one or more selection marker cassettes as described before. The inserted gene is at the locus of the target gene, which is partially or full deleted, in either case being rendered non-functional in that it no longer encodes for an active enzyme.

In host cells that naturally produce ethanol but not 1-butanol, the alcohol dehydrogenase genes produce enzymes that efficiently catalyze the reduction of acetaldehyde to ethanol, but are less efficient at catalyzing the reduction of butyraldehyde to 1-butanol. Accordingly, it is often preferred to introduce an alcohol dehydrogenase gene from a natural 1-butanol producer, even if the wild-type host cells contain one or more alcohol dehydrogenase genes.

The host cell may contain multiple PDC genes as a wild-type strain. Native I. orientalis cells, for example, contain two PDC genes. Other I. orientalis strains, such as ATCC 32196, appear to have two alleles that produce bands of similar size. When the host cell contains multiple PDC genes, it is preferred to delete or disrupt at least one of them and more preferred to disrupt all of them, as this destroys the cell's ability to produce ethanol. Thus, in I. orientalis, it is preferred to disrupt the IoPDC1A or IoPDC1B genes and more preferred to delete or disrupt both alleles of the IoPDC1 locus.

In some embodiments of the process of the invention, ethanol is produced in a yield of 10% or less, preferably in a yield of 2% or less, based on the fermentable sugar. In especially preferred embodiments to maximize the yield of 1-butanol, ethanol is not detectably produced. The invention can also be carried out, however, to co-produce 1-butanol and ethanol, in which case deletion or disruption of PDC or ADH is not necessary or preferable.

The genetically modified yeast cell of the invention may have additional genetic modifications that provide some desired attribute to the cells. A genetic modification(s) of particular interest provides a genetic pathway which permits the cell to more easily metabolize pentose sugars such as xylose. Among such modifications are (1) insertion of a functional exogenous xylose isomerase gene, (2) a deletion or disruption of a native gene that produces an enzyme that catalyzes the conversion of xylose to xylitol, (3) a deletion or disruption of a functional xylitol dehydrogenase gene and/or (4) modifications that cause the cell to overexpress a functional xylulokinase. Methods for introducing such modifications into yeast cells are described, for example, in WO 04/099381, incorporated herein by reference.

In the fermentation process of the invention, the cell of the invention is cultivated in a fermentation medium that includes a carbon source that is fermentable by the transformed cell. Any carbon source that can be fermented by the provided yeast cell can be used. Some preferred carbon sources include sugars such as glucose, xylose, arabinose, sucrose, fructose, cellulose, glucose oligomers, and glycerol. The sugar or sugars to be fermented by the yeast cells of the invention can be obtained from renewable resources such as corn stover, corn fiber, wheat straw, rice straw, sugarcane bagasse, hardwoods, softwoods, pulp and paper wastes, recycled paper, forest residues, and process streams containing any of these materials. Sugars obtained from two or more of these resources can also be used. Other carbon sources that can be fermented by the engineered yeast cells of the invention, either alone or in combination with sugars, include (but are not limited to) glycerol and organic acids such as lactic acid or acetic acid.

The sugar may be a hexose sugar such as glucose, glycan, maltose, maltotriose, isomaltotriose, panose, fructose, and other glucose or fructose oligomers. If the cell is modified to impart an ability to ferment pentose sugars, the fermentation medium may include one or more pentose sugars such as xylose, xylan or other oligomers of xylose. Such pentose sugars are suitably hydrolysates of a hemicellulose-containing biomass. In case of oligomeric sugars, it may be necessary to add enzymes to the fermentation broth in order to digest these to the corresponding monomeric sugar(s) for fermentation by the cell.

The medium will typically contain nutrients as required by the particular cell, including a source of nitrogen (such as amino acids, proteins, inorganic nitrogen sources such as ammonia or ammonium salts, and the like), and various vitamins, minerals and the like. In some embodiments, the cells of the invention can be cultured in a chemically defined medium in which the only nitrogen sources are inorganic materials. However, it is also possible to culture the cells of the invention in a complex medium that is not chemically defined and which may contain organic nitrogen sources such as proteins, partially digested proteins, and/or amino acids.

Other fermentation conditions, such as temperature, cell density, selection of substrate(s), selection of nutrients, and the like are not considered to be critical to the invention and are generally selected to provide an economical process. Temperatures during each of the growth phase and the production phase may range from above the freezing temperature of the medium to about 50° C. A preferred temperature, particularly during the production phase, is from about 30-45° C.

During the production phase, the concentration of cells in the fermentation medium is typically in the range of about 0.1-20, preferably about 0.1-5, even more preferably about 1-3 g dry cells/liter of fermentation medium.

The fermentation may be conducted aerobically, microaerobically, or anaerobically. By “microaerobic” is meant that some oxygen is fed to the fermentation, and the microorganisms take up the oxygen fast enough such that the dissolved oxygen concentration during production of 1-butanol averages less than about 2% of the saturated oxygen concentration under atmospheric air. “Quasi-anaerobic” conditions, in which no oxygen is added during the fermentation but dissolved oxygen is present in the fermentation medium at the start of the fermentation, can also be used. If desired, specific oxygen uptake rate can be used as a process control, as described in U.S. Patent Application Publication No. 20040043444.

When the fermentation broth contains acidic components, such as acetic acid, uronic acid, or other acids either produced by the yeast or present in the starting media, the medium may be buffered during the production phase of the fermentation. Buffering can maintain the pH in a range of about 3.0 to about 7.0, preferably about 4.5 to about 5.5. Suitable buffering agents include, for example, calcium hydroxide, calcium carbonate, sodium hydroxide, potassium hydroxide, potassium carbonate, sodium carbonate, ammonium carbonate, ammonia, ammonium hydroxide, and the like. In general, those buffering agents that have been used in conventional fermentation processes are also suitable here.

The process of the invention can be conducted continuously, batch-wise, or some combination thereof.

In the process, the yield of 1-butanol on the carbon source is preferably at least 10%, 20%, 30%, 40%, 50%, or higher of the theoretical yield. The concentration, or titer, of 1-butanol will be a function of the yield as well as the starting concentration of the carbon source (and time). In some embodiments, the 1-butanol titer reaches at least 10, 20, 30, 40, 50 g/L, or higher, at some point during the fermentation (preferably at the end of the fermentation). In other embodiments, 1-butanol is removed from the fermentation continuously, so that the titer remains lower than of the 1-butanol was not removed continuously.

The 1-butanol produced according to the present invention can be used in any number of ways known commercially. The 1-butanol can be utilized as a liquid transportation or stationary-power fuel, either in substantially pure form or in combination with other liquid fuels such as gasoline, diesel fuel, biodiesel, or ethanol. 1-butanol can be fed to a boiler or other apparatus for generating heat and/or power. 1-butanol can be gasified to produce syngas, and it can be reformed to produce mixtures comprising high concentrations of hydrogen.

The 1-butanol can further be used as a starting material for plastics production and as a solvent, either in substantially pure form or in combination with other liquid solvents or additives.

The invention will now be characterized by describing the construction of various specific plasmids and overexpression constructs, followed by examples of overexpression in yeast cells.

Construction of Plasmid (pCA87) for Targeted Insertion at the I. orientalis CYB2A Locus.

Plasmid pMI454 is described in FIG. 12 of WO 07/117,282. This plasmid is cut with SfoI and SnaBI and a 6.7 kb fragment is gel-purified and ligated to recircularize without the IoCYB2A 3′ flank. This ligation generates plasmid pCA87 as shown in FIG. 1A.

Construction of Plasmid (pCA88) for Targeted Insertion at the I. orientalis CYB2A Locus.

The transcriptional terminator from the IoPDC1 locus (T_{IoPDC1}) is amplified from wild-type I. orientalis genomic DNA using primers identified as SEQ ID NO:1 and SEQ ID NO:2. These primers add an NdeI site on the 5′ of the terminator and an AseI site on the 3′ side. The resulting PCR product is then cut with NdeI and AseI. Plasmid pCA87 (FIG. 1A) is linearized with NdeI. The linearized plasmid and digested PCR fragments are ligated. Restriction mapping is completed to isolate a ligation product with the IoPDC1 terminator sequence in the same orientation as the MEL5 expression cassette. This plasmid is called pCA88 as shown in FIG. 1B.

Construction of Plasmid (pCA92) for Targeted Insertion at the I. orientalis CYB2A Locus.

The sequence upstream of the IoCYB2A locus (IoCYB2A 5′flank) is amplified from wild-type I. orientalis genomic DNA using the primers SEQ ID NO:3 and SEQ ID NO:4. These primers add an AatII site on the 5′ end of the PCR product and an NdeI site on the 3′ side. The resulting PCR product is then cut with AatII and NdeI. Plasmid pCA88 (FIG. 1B) is digested with AatII and NdeI and the 6.5 kb fragment is gel purified. The plasmid fragment and the cut PCR fragment are ligated. Restriction mapping is completed to isolate a ligation product with the IoCYB2A 5′flank sequence in the same orientation as the MEL5 expression cassette. This resulting plasmid is called pCA92 as shown in FIG. 2.

Construction of Plasmid (pCM177) for Targeted Insertion at the I. orientalis CYB2A Locus.

The transcriptional promoter from the IoPDC1 locus (P_{IoPDC1}) is amplified from wild-type I. orientalis genomic DNA using the following primers SEQ ID NO:5 and SEQ ID NO:6. These primers add a SbfI site on the 5′ of the promoter and an NdeI site on the 3′ side. The resulting PCR product is then cut with NdeI and SbfI. Plasmid pCA92 (FIG. 2) is cut with NdeI and SbfI and the linearized plasmid is ligated with the digested PCR product. The resulting plasmid is pCM177 as shown in FIG. 3.

Construction of Plasmid (pCA96) for Targeted Insertion at the I. orientalis CYB2B Locus.

Plasmid pCA87 (FIG. 1A) is cut with SmaI and EcoRI and a 6.2 kb fragment is gel purified. The IoCYB2B 3′flank sequence is amplified from wild-type I. orientalis genomic DNA using the following primers: SEQ ID NO:7 and SEQ ID NO:8. These primers add a SmaI site on the 5′ end of the flanking sequence and an EcoRI site on the 3′ side. The resulting PCR product is then cut with SmaI and EcoRI. The plasmid fragment and PCR fragment are ligated and the resulting plasmid is called pCA96 as shown in FIG. 4.

Construction of Plasmid (pCA97) for Targeted Insertion at the I. orientalis CYB2B Locus.

The transcriptional terminator from the IoPDC1 locus (T_{IoPDC1}) is amplified from wild-type I. orientalis genomic DNA using the following primers: SEQ ID NO:1 and SEQ ID NO:2. These primers add an NdeI site on the 5′ of the terminator and an AseI site on the 3′ side. The resulting PCR product is then cut with NdeI and AseI. Plasmid pCA96 (FIG. 4) is linearized with NdeI. The linearized plasmid and digested PCR fragments are ligated. Restriction mapping is completed to isolate a ligation product with the IoPDC1 terminator sequence in the same orientation as the MEL5 expression cassette. The resulting plasmid is called pCA97 as shown in FIG. 5.

Construction of Plasmid (pCA98) for Targeted Insertion at the I. orientalis CYB2B Locus.

The sequence upstream of the IoCYB2B locus (IoCYB2B 5′flank) is amplified from wild-type I. orientalis genomic DNA using the primers SEQ ID NO:9 and SEQ ID NO:10. These primers add an NdeI site on the 5′ end of the PCR product and an NdeI site on the 3′ side. The resulting PCR product is then cut with NdeI. Plasmid pCA97 (FIG. 5) is digested with NdeI and the vector fragment and the cut PCR fragment are ligated. Restriction mapping is completed to isolate a ligation product with the IoCYB2B 5′ flank sequence in the same orientation as the MEL5 expression cassette. The resulting plasmid is called pCA98 as shown in FIG. 6.

Construction of Plasmid (pCA99) for Targeted Insertion at the I. orientalis CYB2B Locus.

The transcriptional promoter from the IoPDC1 locus (P_{IoPDC1}) is amplified from wild-type I. orientalis genomic DNA using the following primers: SEQ ID NO:51 and SEQ ID NO:6. These primers add an AseI site on the 5′ end of the promoter and an NdeI site on the 3′ side. The resulting PCR product is then cut with NdeI and AseI. Plasmid pCA98 (FIG. 6) is partially digested with NdeI to linearize the vector between the IoCYB2B 5′flank and the T_{IoPDC1}. The linearized plasmid is ligated with the digested PCR product. Restriction mapping is completed to isolate a ligation product with the P_{IoPDC1} PCR fragment in the same orientation as the T_{IoPDC1} sequence. The resulting plasmid is pCA99 as shown in FIG. 7.

Construction of Plasmid (pCA109) for Targeted Insertion at the I. orientalis CYB2B Locus.

Because plasmid pCA99 (FIG. 7) contains two NdeI sites, the NdeI site on the 5′ side of the IoCYB2B 5′flank sequence is removed by site-directed mutagenesis using primers SEQ ID NO:11 and SEQ ID NO:12. The site-directed mutagenesis is completed using the QuikChange Multi Site-Directed Mutagenesis Kit [Stratagene, La Jolla, Calif., USA, product no. 200513]. The resulting plasmid is pCA109 as shown in FIG. 8.

Construction of Plasmid (pCA100) for Targeted Insertion at the I. orientalis GPD1 Locus.

Plasmid pCA87 (FIG. 1A) is cut with SmaI and EcoRI and the 6.2 kb fragment is gel purified. The IoGPD1 3′flank sequence is amplified from wild-type I. orientalis genomic DNA using primers SEQ ID NO:13 and SEQ ID NO:14. These primers add a SmaI site on the 5′ end of the flanking sequence and an EcoRI site on the 3′ side. The resulting PCR product is then cut with SmaI and EcoRI. The plasmid fragment and PCR fragment are ligated and the resulting plasmid is called pCA100 as shown in FIG. 9.

Construction of Plasmid (pCA101) for Targeted Insertion at the I. orientalis GPD1 Locus.

The transcriptional terminator from the IoPDC1 locus (T_{IoPDC1}) is amplified from wild-type I. orientalis genomic DNA using primers identified as SEQ ID NO:1 and SEQ ID NO:2. These primers add an NdeI site on the 5′ side of the terminator and an AseI site on the 3′ side. The resulting PCR product is then cut with NdeI and AseI. Plasmid pCA100 (FIG. 9) is linearized with NdeI. The linearized vector and digested PCR fragments are ligated. Restriction mapping isolates a ligation product with the IoPDC1 terminator sequence in the same orientation as the MEL5 expression cassette. This plasmid is called pCA101 as shown in FIG. 10.

Construction of Plasmid (pCA102) for Targeted Insertion at the I. orientalis GPD1 Locus.

The sequence upstream of the IoGPD1 locus (IoGPD1 5′flank) is amplified from wild-type I. orientalis genomic DNA using primers identified as SEQ ID NO:15 and SEQ ID NO:16. These primers add an AatII site on the 5′ end of the PCR product and an NdeI site on the 3′ side. The resulting PCR product is then cut with AatII and NdeI. Plasmid pCA101 (FIG. 10) is digested with AatII and NdeI and the 6.8 kb fragment is gel purified. The plasmid fragment and the cut PCR fragment are ligated. Restriction mapping is completed to isolate a ligation product with the IoGPD1 5′ flank sequence in the same orientation as the MEL5 expression cassette. The resulting plasmid is called pCA102 as shown in FIG. 11.

Construction of Plasmid (pCA103) for Targeted Insertion at the I. orientalis GPD1 Locus.

The transcriptional promoter from the IoPDC1 locus (P_{IoPDC1}) is amplified from wild-type I. orientalis genomic DNA using the primers identified as SEQ ID NO:51 and SEQ ID NO:6. These primers add an AseI site on the 5′ end of the promoter and an NdeI site on the 3′ side. The resulting PCR product is then cut with NdeI and AseI. Plasmid pCA102 (FIG. 13) is digested with NdeI to linearize the vector. The linearized plasmid is ligated with the digested PCR product. Restriction mapping is completed to isolate a ligation product with the P_{IoPDC1} PCR fragment in the same orientation as the T_{IoPDC1} sequence. The resulting plasmid is pCA103 as shown in FIG. 12.

Construction of Plasmid (pCA104) for Targeted Insertion at the I. orientalis PDC1 Locus.

Plasmid pCA87 (FIG. 1A) is cut with SmaI and EcoRI and the 6.2 kb fragment is gel purified. The IoPDC1 3′flank sequence is amplified from wild-type I. orientalis genomic DNA using primers identified as SEQ ID NO:17 and SEQ ID NO:18. These primers add a SmaI site on the 5′ end of the flanking sequence and an EcoRI site on the 3′ side. The resulting PCR product is then cut with SmaI and EcoRI. The plasmid fragment and PCR fragment are ligated and the resulting plasmid is called pCA104 as shown in FIG. 13.

Construction of Plasmid (pCA105) for Targeted Insertion at the I. orientalis PDC1 Locus.

The transcriptional terminator from the IoPDC1 locus (T_{IoPDC1}) is amplified from wild-type I. orientalis genomic DNA using primers identified as SEQ ID NO:1 and SEQ ID NO:2. These primers add an NdeI site on the 5′ end of the terminator and an AseI site on the 3′ side. The resulting PCR product is then cut with NdeI and AseI. Plasmid pCA104 (FIG. 13) is linearized with NdeI. The linearized plasmid and digested PCR fragments are ligated. Restriction mapping is completed to isolate a ligation product with the IoPDC1 terminator sequence in the same orientation as the MEL5 expression cassette. The resulting plasmid is called pCA105 as shown in FIG. 14.

Construction of Plasmid (pCA106) for Targeted Insertion at the I. orientalis PDC1 Locus.

The transcriptional promoter from the IoPDC1 locus (P_{IoPDC1}) is amplified from wild-type I. orientalis genomic DNA using primers identified as SEQ ID NO:51 and SEQ ID NO:6. These primers add an AseI site on the 5′ end of the promoter and an NdeI site on the 3′ side. The resulting PCR product is then cut with NdeI and AseI. Plasmid pCA105 (FIG. 14) is digested with NdeI to linearize the plasmid, and the linearized plasmid is ligated with the digested PCR product. Restriction mapping isolates a ligation product with the P_{IoPDC1} PCR fragment in the same orientation as the T_{IoPDC1} sequence. The resulting plasmid is called pCA106 as shown in FIG. 15.

Pyruvate Formate Lyase Expression Construct Targeted at the I. orientalis Pdc1 Locus.

The E. coli pyruvate formate lyase enzyme (E.C. 2.3.1.54) is encoded by the gene pflB. The gene sequence given in SEQ ID NO:19 and the protein sequence of the enzyme encoded by this gene is given as SEQ ID NO:20. These sequences are found in the KEGG database (www.genome.ad.jp/dbget-bin/www_bget?eco+b0903).

PCR primers identified as SEQ ID NO:21 and SEQ ID NO:22 are used to amplify the pflB gene using E. coli genomic DNA as the template. The 5′ primer adds 22 bp of homology to the pCA106 plasmid upstream of the ATG translational start of this coding sequence. The 3′ primer adds 23 bp of homology to the pCA106 plasmid including and downstream of the TGA STOP codon. The resulting PCR product is recombined with the pCA106 plasmid using the In-Fusion™ 2.0 Dry-Down PCR Cloning Kit [Clontech, Mountain View, Calif., USA, Cat. No. 639609]. The resulting plasmid contains the pflB coding sequence flanked by the I. orientalis PDC1 promoter and terminator and is named pCA116 (FIG. 16).

Pyruvase Formate Lyase Activating Enzyme Expression Construct Targeted at the I. orientalis PDC1 Locus.

The E. coli pyruvate formate lyase activating enzyme (E.C. 1.97.1.4) is encoded by the gene pflA. The gene sequence is identified as SEQ ID NO:23 and the protein sequence of the enzyme encoded by this gene is identified as SEQ ID NO:24. These sequences are obtained from the KEGG database (www.genome.ad.jp/dbget-bin/www_bget?eco:b0902).

PCR primers identified as SEQ ID NO:25 and SEQ ID NO:26 are used to amplify the pflA gene using E. coli genomic DNA as the template. The 5′ primer adds an NdeI at the ATG translational start of this coding sequence and the 3′ primer converts the STOP codon from a “TAA” to a “TGA” and adds a BclI site. The resulting PCR product is cut with NdeI and BclI. Plasmid pCA106 (FIG. 15) also is cut with NdeI and BclI and then is ligated to the digested PCR product. The resulting plasmid contains the pflA coding sequence flanked by the I. orientalis PDC1 promoter and terminator. It is named pCA117 as shown in FIG. 17.

Acetyl-CoA C-Acetyltransferase Expression Construct for Targeted Insertion at the I. orientalis GPD1 Locus.

The Saccharomyces cerevisiae acetyl-CoA C-acetyltransferase enzyme (E.C. 2.3.1.9) is encoded by the gene ERG10. The gene sequence is identified as SEQ ID NO:27 and the protein sequence for the enzyme encoded by this gene is identified as SEQ ID NO:28. These sequences are obtained from the KEGG database (www.genome.ad.ip/dbget-bin/www_bget?sce+YPL028W).

PCR primers identified as SEQ ID NO:29 and SEQ ID NO:30 are used to amplify the ERG10 gene using S. cerevisiae genomic DNA as the template. The 5′ primer adds an NdeI at the ATG translational start of this coding sequence and the 3′ primer adds a BclI site. The resulting PCR product is cut with NdeI and BclI. Plasmid pCA103 (FIG. 12) also is cut with NdeI and BclI and then is ligated to the digested PCR product. The resulting plasmid contains the ScERG10 coding sequence flanked by the I. orientalis PDC1 promoter and terminator. It is named pCA115 as shown in FIG. 18.

3-Hydroxybutyryl-CoA Dehydrogenase Expression Construct for Targeted Insertion at the I. orientalis CYB2A Locus.

The Clostridium acetobutilicum 3-hydroxybutyryl-CoA dehydrogenase (E.C. 1.1.1.157) enzyme is encoded by the gene hbd. The gene sequence is identified as SEQ ID NO:31 and protein sequence for the enzyme encoded by this gene is identified as SEQ ID NO:32. These are obtained from the KEGG database (www.genome.ad.jp/dbget-bin/www_bget?cac+CAC2708).

PCR primers identified as SEQ ID NO:33 and SEQ ID NO:34 are used to amplify the hcd using Clostridium acetobutilicum genomic DNA as the template. The 5′ primer adds an NdeI at the ATG translational start of this coding sequence and the 3′ primer converts the STOP codon from a “TAA” to a “TGA” and adds a BclI site. The resulting PCR product is cut with NdeI and BclI. Plasmid pCM177 (FIG. 3) also is cut with NdeI and BclI and then is ligated to the digested PCR product. The resulting plasmid contains the hbd coding sequence flanked by the I. orientalis PDC1 promoter and terminator. It is named pCA107 and shown in FIG. 19.

3-Hydroxybutyryl-CoA Dehydratase Expression Construct for Targeted Insertion at the I. orientalis CYB2A Locus.

The Clostridium acetobutilicum 3-hydroxybutyryl-CoA dehydratase (E.C. 4.2.1.55) enzyme is encoded by the gene crt. The gene sequence is identified as SEQ ID NO:35 and the protein sequence of the enzyme encoded by that gene is identified as SEQ ID NO:36. These sequences are obtained from the KEGG database (www.genome.ad.ip/dbget-bin/www_bget?cac+CAC2712).

PCR primers identified as SEQ ID NO:37 and SEQ ID NO:38 are used to amplify the crt using Clostridium acetobutilicum genomic DNA as the template. The 5′ primer adds an NdeI at the ATG translational start of this coding sequence and the 3′ primer converts the STOP codon from a “TAG” to a “TGA” and adds a BclI site. The resulting PCR product is cut with NdeI and BclI. PlasmidpCM177 (FIG. 3) also is cut with NdeI and BclI and then is ligated to the digested PCR product. The resulting plasmid contains the crt coding sequence flanked by the I. orientalis PDC1 promoter and terminator. It is named pCA108 and shown in FIG. 20.

Butyryl-CoA Dehydrogenase (Crotonase) Expression Construct for Targeted Insertion at the I. orientalis CYB2B Locus.

The Clostridium acetobutilicum butyryl-CoA dehydrogenase (crotonase) (E.C. 1.3.99.2) enzyme is encoded by the gene bcd. The gene sequence is identified as SEQ ID NO:39 and the protein sequence for the enzyme encoded by this gene is identified as SEQ ID NO:40. These sequences are obtained from the KEGG database (www.genome.ad.jp/dbget-bin/www_bget?cac+CAC2711).

PCR primers identified as SEQ ID NO:41 and SEQ ID NO:42 are used to amplify the bcd using Clostridium acetobutilicum genomic DNA as the template. The 5′ primer adds an NdeI at the ATG translational start of this coding sequence and the 3′ primer converts the STOP codon from a “TAA” to a “TGA” and adds a BclI site. The resulting PCR product is cut with NdeI and BclI. PlasmidpCA109 (FIG. 8) also is cut with NdeI and BclI and then is ligated to the digested PCR product. The resulting plasmid contains the bcd coding sequence flanked by the I. orientalis PDC1 promoter and terminator. It is named pCA110 and shown in FIG. 21.

Acetaldehyde/Alcohol Dehydrogenase Expression Construct for Targeted Insertion at the I. orientalis CYB2B Locus.

The Clostridium acetobutilicum acaetaldehyde dehydrogenase reaction (E.C. 1.2.1.10) and the alcohol dehydrogenase reaction (E.C. 1.1.1.-) are both encoded by the gene adhE. The gene sequence is identified as SEQ ID NO:43 and the protein sequence of the enzyme encoded by this gene is identified as SEQ ID NO:44. These sequences are obtained from the KEGG database (www.genome.ad.jp/dbget-bin/www_bget?cac+CA_P0035).

PCR primers identified as SEQ ID NO:45 and SEQ ID NO:46 are used to amplify the C-terminal portion of the adhE gene using Clostridium acetobutilicum genomic DNA as the template. The 5′ primer is located at +1634 relative to the ATG and the 3′ primer converts the STOP codon from a “TAA” to a “TGA” and adds a BclI site. The resulting PCR product is cut with NdeI and BclI. PlasmidpCA109 (FIG. 8) also is cut with NdeI and BclI and then is ligated to the digested PCR product. The resulting plasmid contains the C-terminal half of the adhE coding sequence flanked by the I. orientalis PDC1 promoter and terminator. It is named pCA111 as shown in FIG. 22.

PCR primers identified as SEQ ID NO:47 and SEQ ID NO:48 are used to amplify the N-term half of the adhE gene using Clostridium acetobutilicum genomic DNA as the template. The 5′ primer adds an NdeI site at the ATG translational start of this coding sequence and the 3′ primer binds at +2203C relative to the ATG. The resulting product is cut with NdeI and ligated to pCA111 (FIG. 22) linearized with NdeI. Restriction mapping is completed to isolate a ligation product with the N-term adhE PCR fragment in the same orientation as the C-term adhE sequence. The resulting plasmid is named pCA112 as shown in FIG. 23.

Pyruvate Dehydrogenase Expression Construct for Targeted Insetion at the I. orientalis GPD1 Locus.

The Euglena gracilis pyruvate dehydrogenase (1.2.1.51) enzyme is encoded by the gene pno. The gene sequence is identified as SEQ ID NO:49 and protein sequence of the enzyme encoded by that gene is identified as SEQ ID NO:50. These sequences are obtained from GENBANK (Accession No. AJ278425). This gene is synthesis for preparation of an expression construct, with destruction of an NdeI site at +2647 by from the ATG start codon by altering the T at position +2651 to a C. The gene is then cloned onto the NdeI and BclI sites of plasmid pCA103 (FIG. 12). The resulting plasmid contains the pno coding sequence flanked by the I. orientalis PDC1 promoter and terminator regions. It is named pCA125 and is shown in FIG. 24.

### EXAMPLE 1

### Insertion of hbd Gene into an I. orientalis Strain

Plasmid pCA107 (FIG. 19) is digested with BamHI and PspOMI and a 6.6 kb fragment is gel purified. A wild-type I. orientalis strain ATCC PTA-6658 is transformed with 1 μg of this pCA107 fragment using standard lithium acetate methods. Transformed strains generate blue colonies on YNBMel+X-α-Gal (Yeast Nitrogen Base Media with 2% Melibiose as the sole carbon source and X-α-Gal color indicator) plates. The replacement of one allele of the endogenous IoCYB2A locus with the hbd gene is confirmed by PCR and Southern analysis.

The ScMEL5 marker is looped out from one of the transformants via a homologous recombination event through the K. thermotolerans repeat sequences (KtSEQ1). Loop-out strains are white on YPD (Yeast Peptone Dextrose) plates containing the X-α-Gal color indicator. This strain is called NW01.

### EXAMPLE 2

### Insertion of crt Gene into the Strain from Example 1

Plasmid pCA108 (FIG. 20) is digested with BamHI and PspOMI and a 6.6 kb is gel purified. Strain NW01 (from Example 1) is transformed with 1 μg of this pCA108 fragment using standard lithium acetate methods. Transformed strains generate blue colonies on YNBMel+X-α-Gal plates. The replacement of the second allele of the endogenous IoCYB2A locus with the crt gene is confirmed by PCR and Southern analysis. In addition, it is confirmed that the hbd expression cassette remains intact at the other IoCYB2A allele.

The ScMEL5 marker is looped out from one of the transformants via a homologous recombination event through the K. thermotolerans repeat sequences. Loop-out strains are white on YPD (Yeast Peptone Dextrose) plates containing the X-α-Gal color indicator. This strain is called NW02.

### EXAMPLE 3

### Insertion of bcd Gene into the Strain from Example 2

Plasmid pCA110 (FIG. 21) is digested with PfoI and SapI and an 8.1 kb fragment is gel purified. Strain NW02 (Example 2) is transformed with 1 μg of this pCA110 fragment using standard lithium acetate methods. Transformed strains generate blue colonies on YNBMel+X-α-Gal plates. The replacement of the first allele of the endogenous IoCYB2B locus with the bcd gene is confirmed by PCR and Southern analysis.

The ScMEL5 marker is looped out from one of those transformants via a homologous recombination event through the K. thermotolerans repeat sequences. Loop-out strains are white on YPD (Yeast Peptone Dextrose) plates containing the X-α-Gal color indicator. This strain is called NW03.

### EXAMPLE 4

### Insertion of adhE Gene into the Strain of Example 3

Vector pCA112 (FIG. 23) is digested with PfoI and SapI and a 9.8 kb fragment is gel purified. Strain NW03 (Example 3) is transformed with 1 μg of this pCA112 fragment using standard lithium acetate methods. Transformed strains will generate blue colonies on YNBMel+X-α-Gal plates. The replacement of the second allele of the endogenous IoCYB2B locus with the adhE gene is confirmed by PCR and Southern analysis. In addition, it is confirmed that the bcd expression cassette remains intact at the other IoCYB2B allele.

The ScMEL5 marker is looped out from one of the transformants via a homologous recombination event through the K. thermotolerans repeat sequences. Loop-out strains are white on YPD (Yeast Peptone Dextrose) plates containing the X-α-Gal color indicator. This strain is called NW04, and contains the hbd, crt, bcd, and adhE gene cassettes.

### EXAMPLE 5

### Construction of a Mutant I. orientalis Strain that Contains the hbd, crt, bcd, and adhE Genes and has a Deletion of Both Alleles of the PDC1 Locus

Plasmid pCA106 (FIG. 15) is digested with EcoRI and PfoI and a 5.5 kb fragment is gel purified. Strain NW04 (Example 4) is transformed with 1 μg of the resulting fragment using standard lithium acetate methods. Transformed strains generate blue colonies on YNBMel+X-α-Gal plates. The replacement of one allele of the endogenous IoPDC1 locus with the ScMEL5 marker cassette is confirmed by PCR and Southern analysis. The ScMEL5 marker is looped out from one of the transformants via a homologous recombination event through the K. thermotolerans repeat sequences, whereby the loop-out strains are white on YPD plates containing X-α-Gal. This strain is called NW05.

Strain NW05 is again transformed with 1 μg of the same 5.5 kb EcoRI/PfoI pCA106 fragment using standard lithium acetate methods. The replacement of the second allele of the endogenous IoPDC1 locus with the ScMEL5 marker cassette is confirmed by PCR and Southern analysis. In addition, it is confirmed that the pdc1-1Δ containing a single copy of the KtSEQ1 repeat remains intact at the other IoPDC1 allele. The ScMEL5 marker is looped out from one of the transformants via a homologous recombination event through the K. thermotolerans repeat sequences, whereby the loop-out strains are white on YPD plates containing X-α-Gal. This strain is called NW06.

### EXAMPLE 6

### Construction of a Mutant I. orientalis Strain Containing the ERG10, hbd, crt, bcd, and adhE Genes

Plasmid pCA115 (FIG. 18) is digested with PvuI and SapI and a 7.4 kb fragment is gel purified. Strain NW04 (Example 4) is transformed with 1 μg of this fragment using standard lithium acetate methods. Transformed strains generate blue colonies on YNBMel+X-α-Gal plates. The replacement of the first allele of the endogenous IoGPD1 locus with the ScERG10 gene is confirmed by PCR and Southern analysis. The ScMEL5 marker is looped out from one of the transformants via a homologous recombination event through the K. thermotolerans repeat sequences, whereby the loop-out strains are white on YPD plates containing X-α-Gal. This strain is called NW07.

### EXAMPLE 7

### Construction of a Mutant I. orientalis Strain NW08 which Contains the ERG10, hbd, crt, bcd, adhE and pno Genes

Plasmid pCA125 (FIG. 24) is digested with PvuI and EcoRI and an 11.4 kb fragment is gel purified. Strain NW07 (Example 6) is transformed with 1 μg of this fragment using standard lithium acetate methods. Transformed strains generate blue colonies on YNBMel+X-α-Gal plates. The replacement of the second allele of the endogenous IoGPD1 locus with the pno gene is confirmed by PCR and Southern analysis. In addition, it is verified that the ScERG10 expression cassette remains intact at the other IoGPD1 allele. The ScMEL5 marker is looped out from one of the transformants via a homologous recombination event through the K. thermotolerans repeat sequences, whereby the loop-out strains are white on YPD plates containing X-α-Gal. This strain is called NW08.

### EXAMPLE 8

### Construction of Mutant I. orientalis Strain which Contains the hbd, crt, bcd, adhE and pno Genes and the Deletion of Both Alleles of the PDC1 Locus

Plasmid pCA106 (FIG. 15) is digested with EcoRI and PfoI and a 5.5 kb fragment is gel purified. Strain NW08 (Example 7) is transformed with 1 μg of this fragment using standard lithium acetate methods. Transformed strains generate blue colonies on YNBMel+X-α-Gal plates. The replacement of one allele of the endogenous IoPDC1 locus with the ScMEL5 marker cassette is confirmed by PCR and Southern analysis. The ScMEL5 marker is looped out from one of the transformants via a homologous recombination event through the K. thermotolerans repeat sequences, whereby the loop-out strains are white on YPD plates containing X-α-Gal. This strain is called NW09.

Strain NW09 is again transformed with 1 μg of the same 5.5 kb fragment using standard lithium acetate methods. Transformed strains generate blue colonies on YNBMel+X-α-Gal plates. The replacement of the second allele of the endogenous IoPDC1 locus with the ScMEL5 marker cassette is confirmed by PCR and Southern analysis. In addition, it is verified that the pdc1-1Δ containing a single copy of the KtSEQ1 repeat remains intact at the other IoPDC1 allele. The ScMEL5 marker is looped out from one of the transformants via a homologous recombination event through the K. thermotolerans repeat sequences, whereby the loop-out strains are white on YPD plates containing X-α-Gal. This strain is called NW10.

### EXAMPLE 9

### Construction of Mutant I. orientalis Strain which Includes Overexpression of pflA, pflB, hbd, crt, bcd, adhE, and ERG10 Genes, and Deletion of Both Alleles of the PDC1 Locus

Plasmid pCA116 (FIG. 16) is digested with XmnI and EcoRI and an 8.2 kb fragment is gel purified. Strain NW07 (Example 6) is transformed with 1 μg of this fragment using standard lithium acetate methods. Transformed strains generate blue colonies on YNBMel+X-α-Gal plates. The replacement of one allele of the endogenous IoPDC1 locus with the pflB gene is confirmed by PCR and Southern analysis. The ScMEL5 marker is looped out from one of the transformants via a homologous recombination event through the K. thermotolerans repeat sequences (KtSEQ1), whereby the loop-out strains are detectably white on YPD plates containing X-α-Gal. This strain is called NW11.

Plasmid pCA117 (FIG. 17) is digested with PfoI and EcoRI and a 6.2 kb fragment is gel purified. Strain NW11 is transformed with 1 μg of this pCA117 fragment using standard lithium acetate methods. Transformed strains generate blue colonies on YNBMel+X-α-Gal plates. The replacement of the second allele of the endogenous IoPDC1 locus with the pflA gene is verified by PCR and Southern analysis. In addition, it is confirmed that the pflB expression cassette remains intact at the other IoPDC1 allele. The ScMEL5 marker is looped out from one of the transformants via a homologous recombination event through the K. thermotolerans repeat sequences, whereby the loop-out strains are detectably white on YPD plates containing X-α-Gal. This strain is called NW12.

In this detailed description, reference has been made to multiple embodiments and to the accompanying examples and drawings in which is shown by way of illustration specific exemplary embodiments in which the invention may be practiced. Other embodiments that do not provide all of the features and advantages set forth herein may be utilized, without departing from the spirit and scope of the present invention. This invention incorporates routine experimentation and optimization of the methods, apparatus, and systems described herein. Such modifications and variations are considered to be within the scope of the invention defined by the appended claims.

1. A genetically modified yeast cell having an active metabolic pathway from pyruvate to 1-butanol, wherein the active metabolic pathway from pyruvate to 1-butanol includes the reactions a) pyruvate to acetyl-CoA, b) acetyl-CoA to acetoacetyl-CoA, c) acetoacetyl-CoA to 3-hydroxybutanoyl-CoA, d) 3-hydroxybutanoyl-CoA to crotonoyl-CoA, d) crotonoyl-CoA to butyryl-CoA, f) butyryl-CoA to butyraldehyde, and g) butyraldehyde to 1-butanol.

2-4. (canceled)

5. The genetically modified yeast cell of claim 1, which contains at least one active, exogenous gene that produces an enzyme that catalyzes the reaction of pyruvate to acetyl-CoA.

6. The genetically modified yeast cell of claim 5, wherein the exogenous gene is a pyruvate formate lyase (pfl) gene or a pyruvate dehydrogenase (pdh) gene.

7-9. (canceled)

10. The genetically modified yeast cell of claim 5, which contains at least one active, exogenous gene that produces an enzyme that catalyzes the reaction of acetyl-CoA to acetoacetyl-CoA.

11. The genetically modified yeast cell of claim 10, wherein the exogenous gene that produces an enzyme that catalyzes the reaction of acetyl-CoA to acetoacetyl-CoA is an acetyl-CoA C-acetyltransferase gene.

12. (canceled)

13. The genetically modified yeast cell of claim 5, which contains at least one active, exogenous gene that produces an enzyme that catalyzes the reaction of acetoacetyl-CoA to 3-hydroxybutanoyl-CoA.

14. The genetically modified yeast cell of claim 13, wherein the exogenous gene that produces an enzyme that catalyzes the reaction of acetoacetyl-CoA to 3-hydroxybutanoyl-CoA is an acetyl-3-hydroxylbutyryl-COA dehydrogenase (hbd) gene.

15. (canceled)

16. The genetically modified yeast cell claim 5, which contains at least one active, exogenous gene that produces an enzyme that catalyzes the reaction of 3-hydroxybutanoyl-CoA to crotonoyl-CoA.

17. The genetically modified yeast cell of claim 16, wherein the exogenous gene that produces an enzyme that catalyzes the reaction of 3-hydroxybutanoyl-CoA to crotonoyl-CoA is a 3-hydroxybutyryl-CoA dehydratase gene.

18. (canceled)

19. The genetically modified yeast cell of claim 5, which contains at least one active, exogenous gene that produces an enzyme that catalyzes the reaction of crotonoyl-CoA to butyryl-CoA.

20. The genetically modified yeast cell of claim 19, wherein the exogenous gene that produces an enzyme that catalyzes the reaction of crotonoyl-CoA to butyryl-CoA is a 3-butyryl-CoA dehydrogenase (bcd) gene.

21. (canceled)

22. The genetically modified yeast cell of claim 5, which contains at least one active, exogenous gene that produces an enzyme that catalyzes the reaction of butyryl-CoA to butyraldehyde.

23. The genetically modified yeast cell of claim 22, wherein the exogenous gene that produces an enzyme that catalyzes the reaction of butyryl-CoA to butyraldehyde is a butyryl-CoA dehydrogenase gene.

24. (canceled)

25. The genetically modified yeast cell of claim 5, which contains at least one active, exogenous gene that produces an enzyme that catalyzes the reaction of butyraldehyde to 1-butanol.

26. The genetically modified yeast cell of claim 25, wherein the exogenous gene that produces an enzyme that catalyzes the reaction of butyraldehyde to 1-butanol is a 1-butanol dehydrogenase gene.

27. (canceled)

28. The genetically modified yeast cell of claim 5, which overexpresses at least one native gene that catalyzes at least one of reactions a)-g).

29. The yeast cell of claim 3, which further has deletion or disruption of a native pathway from pyruvate to ethanol.

30. (canceled)

31. The yeast cell of claim 29, which further has deletion or disruption of one or more native alcohol dehydrogenase genes.

32. The yeast cell of claim 3, wherein the yeast is of the genera Saccharomyces, Candida, Pichia, Kluyveromyces, Issatchenkia, Yarrowia, Rhodotorula, Hansenula, Schizochytrium, or Thraustochytrium.

33. The yeast cell of claim 32, wherein the yeast species is selected from the group consisting of Issatchenkia orientalis, Hansenula ofunaensis, H. polymorphs, H. anomala, Schizochytrium limacinum, Thraustochytrium striatum, T. roseum, and T. aureum.

34. The yeast cell of claim 33, wherein the yeast species is Issatchenkia orientalis.

35. A process for making 1-butanol, comprising culturing a yeast cell of claim 3 in the presence of a fermentable carbon source under conditions such that the yeast cells metabolize the carbon source and convert at least a portion of the carbon source to 1-butanol.

36. The process of claim 35, wherein ethanol is produced in a yield of 10% or less.

37. (canceled)

38. The process of claim 36, wherein ethanol is not detectably produced.

39. The process of any claim 35, wherein at least one carbon source is selected from the group consisting of glucose, xylose, arabinose, sucrose, fructose, cellulose, glucose oligomers, and glycerol.

40. (canceled)

41. The process of claim 35, wherein the final yield of 1-butanol on the carbon source is at least 50% of theoretical.

42. (canceled)

43. The process of claim 41, wherein the broth titer of 1-butanol reaches at least 50 g/L.

44. (canceled)
